(19) United States
(12) Patent Application Publication
(10) Pub. No.: US 2025/0352074 A1
(43) Pub. Date: Nov. 20, 2025

(54) STEERING WHEEL SENSORY SYSTEM THAT DETECTS HEART RATE OF A VEHICLE DRIVER

(71) Applicant: Ahmad Na'eem Shabazz, Claymont, DE (US)

(72) Inventor: Ahmad Na'eem Shabazz, Claymont, DE (US)

(21) Appl. No.: 19/094,531

(22) Filed: Mar. 28, 2025

Related U.S. Application Data
(60) Provisional application No. 63/648,279, filed on May 16, 2024.

(51) Int. Cl. A61B 5/0205; A61B 5/00; A61B 5/024; A61B 5/1455; B60K 35/22; B60K 35/28; B60K 35/60; B60R 25/021; B60R 25/08; B62D 1/04; G06F 3/0482; G06F 3/0487

(52) U.S. Cl. CPC A61B 5/0205; A61B 5/14551; A61B 5/6893; A61B 5/742; B60K 35/22; B60K 35/28; B60K 35/60; B60R 25/021; B60R 25/08; B62D 1/046; A61B 5/024; A61B 2503/22; A61B 2562/166; B60K 2360/11; B60K 2360/16; B60K 2360/21; B60K 2360/33; B60K 2360/332; B60K 2360/782; G06F 3/0482; G06F 3/0487

(57) ABSTRACT

A steering wheel sensory system is disclosed. The steering wheel sensory system is embedded in a steering wheel of a vehicle and detects heart rate of a driver when the hands of the driver are placed on the steering wheel. The steering wheel sensory system includes several components including a vehicle slowdown mechanism, a control system, and a sensor (or sensors) that can detect the heart rate of the driver when their hands are placed on the steering wheel. In this way, the steering wheel sensory system monitors the user's heart rate during operation of the vehicle, whether driving or while parked (but turned on). When erratic heart rhythms are detected due to any cause (e.g., road rage, intoxication, cardiovascular disease, congestive heart failure, arrhythmia, heart attack, stroke, etc.), the steering wheel sensory system triggers a process to slow the vehicle down safely and prompt for further needs.

### CLAIM OF BENEFIT TO PRIOR APPLICATION

This application claims benefit to U.S. Provisional Patent Application 63/648,279, entitled “STEERING WHEEL SENSOR SAFETY SYSTEMS,” filed May 16, 2024. The U.S. Provisional Patent Application 63/648,279 is incorporated herein by reference.

### BACKGROUND

Embodiments of the invention described in this specification relate generally to vehicle safety systems, and more particularly, to a steering wheel sensory system that is embedded in a steering wheel of a vehicle and detects heart rate of a driver when the hands of the driver are placed on the steering wheel.

Many people drive vehicles in modern society between locations for work and/or pleasure. Vehicles often travel at a high rate of speed and can be dangerous if not properly driven. Even if individuals are driving safely, they may experience health problems that may put themselves and others in danger. A suitable means for detecting an unhealthy condition of a driver is desirable.

One particular type of problem many drivers may knowingly or unknowingly have relates directly to their cardiovascular health. In the population as a whole, cardiovascular health tends to diminish as people get older. While many older drivers have excellent cardiovascular health, the fact remains that cardiovascular disease (e.g., congestive heart failure, arrhythmia, heart attack, stroke, etc.) is the number one killer, year in and year out. Thus, the older a driver gets, the more likely the driver will be at risk of a cardiovascular health problem, such as ischemic stroke or myocardial infarction, or other conditions triggered by blood clots or vasoconstriction. But even younger drivers can have cardiovascular health conditions which go undetected until a problem arises. In fact, cardiovascular health events often arise without warning. Given the relative unpredictability of when cardiovascular problems may occur, the potential for car accidents due to cardiovascular health of drivers is a major concern (both for the driver and others).

Beyond the underlying cardiovascular health concerns noted above, many drivers are prone to emotional triggers while driving. For some drivers in some situations, the emotions triggered while driving may be sudden and full of rage. Such emotions while driving are commonly referred to as “road rage.” Road rage, in and of itself, may indicate that the driver is not in condition to drive safely. In addition, road rage is such a strong emotional experience that it is likely to have an effect on the heart and vascular system of the driver, possibly to the point of triggering a physiological event that impacts the cardiovascular health of the driver. Thus, road rage and other strong emotional states of drivers are a concern for the health of the driver and others.

Currently, only drivers who are aware of any underlying cardiovascular disease or related cardiovascular health conditions they may have are able to do anything preventative in regards to driving. For instance, a driver with a known cardiovascular health condition may be prescribed a medication that reduces their risk of suffering from a cardiovascular health event, whether driving or not. Also, some drivers who experience road rage may have self-awareness of this and may be better prepared to take a safe driving action, such as slowing down or pulling off the side of the road, when the strong emotions are felt by the driver. Nevertheless, the vast majority of drivers are still prone to the unpredictable problems that may arise while driving-whether cardiovascular health-driven problems or emotion-driven problems. Even the drivers with known issues cannot predict if or when a cardiovascular health problem may arise.

On the other hand, various physiological markers are detectable in drivers. For instance, a sudden increase in heart rate is a detectable physiological marker that corresponds to road rage in drivers. Similarly, erratic heart rhythms may indicated road rage or intoxication of the driver, or may even indicate that the driver is presently experiencing a cardiovascular health event.

Therefore, what is needed is a way to detect, in a driver and while driving, certain physiological markers that indicate cardiovascular health problems or strong emotional states, such as road rage, which may increase driving risk.

### BRIEF DESCRIPTION

A novel steering wheel sensory system is disclosed. In some embodiments, the steering wheel sensory system is embedded in a steering wheel of a vehicle and detects heart rate of a driver when the hands of the driver are placed on the steering wheel. In some embodiments, the steering wheel sensory system monitors the user's heart rate during operation of the vehicle. If and when the steering wheel sensory system detects any erratic heart rhythms due to any cause (e.g., road rage, intoxication, cardiovascular disease, congestive heart failure, arrhythmia, heart attack, stroke, etc.), the steering wheel sensory system triggers a process to slow the vehicle down safely. The steering wheel sensory system also prompts the user (who is the driver) to pull over and gather oneself and/or asks the driver whether medical attention is requested or needed.

In some embodiments, the steering wheel sensory system comprises (i) a vehicle slowdown mechanism linked to the anti-locking braking (ABS) system of the vehicle, (ii) an electronic system, such as a controller, a micro-controller, a computer system, etc., and (iii) a sensor that can detect the heart rate of the driver when the drive places their hands on the steering wheel. In some embodiments, the sensor comprises an integrated pulse oximeter and heart rate monitor sensor that measures the oxygen level and heart rate of the driver while the driver's hands are placed on the steering wheel. In some embodiments, the electronic system is configured to receive the heart rate and pulse oximeter data captured by the integrated pulse oximeter and heart rate monitor sensor and determine whether the driver is experiencing or is at risk of suffering from a cardiovascular health problem that could negatively impact the driving ability of the driver.

In some embodiments, the steering wheel sensory system further comprises a display screen positioned in the center of the steering wheel and configured to visually output the heart rate and pulse oximeter data captured by the integrated pulse oximeter and heart rate monitor sensor. In some embodiments, the steering wheel sensory system further comprises directional controls or toggle buttons that enables interaction and selection within a menu visually output onto the display screen. In some embodiments, the steering wheel sensory system further comprises a sensor hub that encapsulates the electronic system, the integrated pulse oximeter and heart rate monitor sensor, and wiring that connects the components together. In some embodiments, the display screen, the directional controls, and the toggle buttons are exposed along an outer surface of the sensor hub. In some embodiments, the steering wheel sensory system further comprises a camera the capture facial imagery or video of the driver while driving. In some embodiments, the camera is wired to the electronic system and transmits the imagery or streams the video to the electronic system for processing in real-time as the driver operates the vehicle.

The preceding Summary is intended to serve as a brief introduction to some embodiments of the invention. It is not meant to be an introduction or overview of all inventive subject matter disclosed in this specification. The Detailed Description that follows and the Drawings that are referred to in the Detailed Description will further describe the embodiments described in the Summary as well as other embodiments. Accordingly, to understand all the embodiments described by this document, a full review of the Summary, Detailed Description, and Drawings is needed. Moreover, the claimed subject matters are not to be limited by the illustrative details in the Summary, Detailed Description, and Drawings, but rather are to be defined by the appended claims, because the claimed subject matter can be embodied in other specific forms without departing from the spirit of the subject matter.

### BRIEF DESCRIPTION OF THE DRAWINGS

Having described the invention in general terms, reference is now made to the accompanying drawings, which are not necessarily drawn to scale, and wherein:

FIG. 1 conceptually illustrates a front view of a steering wheel sensory system integrated into a first type of steering wheel in some embodiments.

FIG. 2 conceptually illustrates a front view of the steering wheel sensory system integrated into a second type of steering wheel in some embodiments.

FIG. 3 conceptually illustrates a side perspective view of the steering wheel sensory system integrated into the first type of steering wheel in some embodiments.

FIG. 4 conceptually illustrates a side perspective view of the steering wheel sensory system integrated into the second type of steering wheel in some embodiments.

FIG. 5 conceptually illustrates a side view of the axle/spindle for rotating the first type of steering wheel in some embodiments.

FIG. 6 conceptually illustrates a side view of the axle/spindle for rotating the second type of steering wheel in some embodiments.

FIG. 7 conceptually illustrates a front view of the axle/spindle in the middle of the first type of steering wheel in some embodiments.

FIG. 8 conceptually illustrates a front view of the axle/spindle of the second type of steering wheel in some embodiments.

FIG. 9 conceptually illustrates an interior view of the components of the steering wheel sensory system embedded in the second type of steering wheel in some embodiments.

FIG. 10 conceptually illustrates an electronic system with which some embodiments of the invention are implemented.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Embodiments of the invention described in this specification include a steering wheel sensory system that is embedded in a steering wheel of a vehicle and detects heart rate of a driver when the hands of the driver are placed on the steering wheel. In some embodiments, the steering wheel sensory system monitors the user's heart rate during operation of the vehicle. If and when the steering wheel sensory system detects any erratic heart rhythms due to any cause (e.g., road rage, intoxication, cardiovascular disease, congestive heart failure, arrhythmia, heart attack, stroke, etc.), the steering wheel sensory system triggers a process to slow the vehicle down safely. The steering wheel sensory system also prompts the user (who is the driver) to pull over and gather oneself and/or asks the driver whether medical attention is requested or needed.

In some embodiments, the steering wheel sensory system comprises (i) a vehicle slowdown mechanism linked to the anti-locking braking (ABS) system of the vehicle, (ii) an electronic system, such as a controller, a micro-controller, a computer system, etc., and (iii) a sensor that can detect the heart rate of the driver (“user”) when the user places their hands on the steering wheel.

In some embodiments, the steering wheel sensory system further comprises a sensor hub that is centrally positioned within the steering wheel and includes the one or more sensor(s).

In some embodiments, the steering wheel sensory system further comprises a display screen integrated into and exposed along an exterior surface of the sensor hub and configured to visually output a menu other display options including pulse, oxygen, and heart rate numbers detected by the steering wheel sensory system for benefit of the driver and/or passengers in the vehicle. In some embodiments, the display screen comprises a liquid crystal display (LCD). In some embodiments, the display screen comprises a light emitting diode (LED) display screen. In some embodiments, the display screen comprises an organic light emitting diode (OLED) display screen.

In some embodiments, the steering wheel sensory system further comprises one or more directional controllers that are configured to control a menu and other display options visually output on the display screen.

In some embodiments, the steering wheel sensory system further comprises toggle buttons that provide directional control for controlling the menu and other display options visually output on the display screen of the steering wheel sensory system, in the absence of directional controllers for the steering wheel.

In some embodiments, the steering wheel sensory system further comprises a camera integrated into the steering wheel sensory system, exposed along the exterior surface of the sensor hub, and configured to track the driver's facial status when driving.

In some embodiments, the steering wheel sensory system further comprises an integrated pulse oximeter and heart rate monitor sensor component that measures the oxygen level and heart rate of the driver and is embedded within the sensor hub.

In some embodiments, the electronic system comprises a control circuit (or “controller”) that provides data processing of sensor data, performs bypass detection, and operates as the command center of the steering wheel sensory system. In some embodiments, the controller comprises a printed circuit board (PCB). In some embodiments, the PCB comprises a micro-controller. In some embodiments, the PCB comprises a processor, a memory, and a sensor bus. In some embodiments, the sensor data bus comprises a Universal Asynchronous Receiver Transmitter (UART) bus. In some embodiments, the sensor data bus comprises an Inter-Integrated Circuit (I2C) bus. In some embodiments, the sensor data bus comprises a Serial Peripheral Interface (SPI) bus. In some embodiments, the sensor data bus comprises a Controller Area Network (CAN) bus. In some embodiments, the PCB further comprises a wireless communication module. In some embodiments, the wireless communications module comprises a Bluetooth® communications device. In some embodiments, the wireless communications module comprises a WiFi device. In some embodiments, the wireless communications module comprises a cellular communications device. In some embodiments, the wireless communications module comprises a global positioning system (GPS) device.

In some embodiments, the PCB is a computing device. In some embodiments, the computing device comprises a single board computer (SBC) with the processor, the memory, and the bus. In some embodiments, the processor comprises a central processing unit (CPU). In some embodiments, the processor comprises a graphics processing unit (GPU). In some embodiments, the processor comprises an artificial intelligence (AI) processor. In some embodiments, the processor comprises a neural processing unit (NPU).

In some embodiments, the sensor is a first sensor and the steering wheel sensory system further comprises a plurality of sensors including the first sensor. In some embodiments, the sensors are light photo sensors which capture sensor data including the heart rate and oxygen levels of the driver.

In some embodiments, the steering wheel sensory system further comprises wiring that interconnects the components of the steering wheel sensory system, providing control, power, and communicative channels between the components for operation of the steering wheel sensory system. By interconnecting the components with the wiring, the plurality of light photo sensors are able to send the sensor data (after capturing the heart rate and oxygen levels of the driver) to the PCB controller of the steering wheel sensory system which, in turn, evaluates the sensor data to determine whether to slow down and stop the vehicle due to a detected cardiovascular health event or allow the driver to continue driving as normal. Also, in some embodiments of the steering wheel sensory system, the plurality of light photo sensors are embedded within the steering wheel felloe onto which the hands of the driver are placed during operation of the vehicle.

In some embodiments, the vehicle slowdown mechanism comprises a wired connection between the PCB controller and the vehicle's anti-locking braking system (ABS). Thus, when the processor of the PCB controller evaluates the captured sensor data (that is, the heart rate and oxygen levels of the driver) and detects a present cardiovascular health event or a high risk of the driver experiencing a cardiovascular health problem while driving, the PCB controller sends a signal command to the ABS to start applying the brakes of the vehicle to slow down the speed of the vehicle, eventually to a stop. While this is happening, the PCB controller also sends alert notifications to the driver via audible alarms (beeping or ringing) and visual notifications visually output on the display screen integrated into the sensor hub of the steering wheel.

As stated above, currently, only drivers who are aware of any underlying cardiovascular disease or condition they may have are able to do anything preventative in regards to driving (e.g., taking their medication as prescribed). Also, some drivers who experience road rage may have self-awareness of this and may be better prepared to take a safe driving action, such as slowing down or pulling off the side of the road, when the strong emotions are felt by the driver. Nevertheless, the vast majority of drivers are still prone to the unpredictable problems that may arise while driving-whether cardiovascular health-driven problems or emotion-driven problems. Even the drivers with known issues cannot predict if or when a cardiovascular health problem may arise. Yet, we have the ability to detect various physiological markers of individuals, including heart and cardiovascular markers of drivers. For instance, a sudden increase in heart rate is a detectable physiological marker that may correspond to road rage in drivers. Similarly, erratic heart rhythms may indicated road rage or intoxication of the driver, or may even indicate that the driver is presently experiencing a cardiovascular health event. Embodiments of the steering wheel sensory system described in this specification solve such problems by embedding a sensor system into the steering wheel of a vehicle, such that when a driver places their hands on the steering wheel, the sensor system is able to detect, in real-time while the driver is operating the vehicle, various physiological markers of cardiovascular health of the driver. Furthermore, the steering wheel sensory system links into the vehicle's anti-locking braking system (ABS) which enables the steering wheel sensory system to slow the speed of the vehicle down when a cardiovascular event, such as an erratic heartbeat, is detected. In addition, the steering wheel sensory system will detect an erratic heartbeat due to strong emotions, such as experienced during road rage, and will prompt the driver of vehicle to slow down and pull over safely.

Embodiments of the steering wheel sensory system described in this specification differ from and improve upon currently existing options. In particular, the steering wheel sensory system improves safety and awareness in regards to driving while having cardiovascular disease and can be used as a preventative method for early diagnosis. For example, high blood pressure may be an early indication that a driver has started developing cardiovascular health issues which could give rise to stroke, heart attack, etc., while driving. Although high blood pressure often has no symptoms, over time, if untreated, it can cause serious health issues. Thus, the steering wheel sensory system can also provide a proactive way to identify possible risk factors and/or be a preventative method for getting a medical diagnosis.

In some embodiments, the steering wheel sensory system is preventative in starting the vehicle for operation. As a person of ordinary skill in the art would appreciate, modern vehicles are designed to lock the steering wheel when the driver shuts off the ignition. Similarly, the steering wheel sensory system described in this specification is designed to maintain the locked steering wheel upon starting up the vehicle. In some embodiments, the steering wheel sensory system prevents the steering wheel from unlocking until it has initialized and established a heart rate of the driver. Thus, the driver would need to place his or her hands on the steering wheel and give the steering wheel sensory system a moment to capture a baseline heart rate. In some embodiments, the steering wheel sensory system comprises a bypass detection system that triggers an alarm if and when the driver puts on a protective covering over their hands (especially, gloves). Some drivers may attempt to “bypass” the initialization sequence of obtaining the baseline heart rate by putting on gloves that can obscure the ability of the steering wheel sensory system to detect heart rate. In that case, the steering wheel sensory system will trigger an audible alarm to sound. For example, a beeping alarm noise similar to conventional driver seat belt alarms which ring when the driver does not fasten their seat belt during operation of the vehicle.

The steering wheel sensory system is equipped with components that are suitable for any type of vehicular steering wheel. Thus, the steering wheel sensory system can be utilized by anyone who operates a motor vehicle, including drivers with a commercial drivers license (CDL), which is the type of driver's license required in the United States to operate large and heavy vehicles (including trucks, buses, and trailers) or a vehicle of any size that transports hazardous materials or cargo. Not only will the steering wheel sensory system help save lives among passenger vehicle drivers, CDL drivers, occupants in the driver's vehicle, occupants in nearby vehicles, and pedestrians, it may also help individuals save money on car insurance. For instance, car insurers may identify lower risk profiles of drivers whose vehicles are equipped with the steering wheel sensory system.

The steering wheel sensory system of the present disclosure may be comprised of the following elements. This list of possible constituent elements is intended to be exemplary only and it is not intended that this list be used to limit the steering wheel sensory system of the present application to just these elements. Persons having ordinary skill in the art relevant to the present disclosure may understand there to be equivalent elements that may be substituted within the present disclosure without changing the essential function or operation of the steering wheel sensory system.

1. A directional controller that controls options in the menu shown on the display screen (e.g., LCD, LED, OLED display screen, etc.), where the menu has display configuration options as well as audible alert options and other user-selectable options to configure operation of the steering wheel sensory system and, especially, the visual output presentation of the sensor-based data-namely, the heart rate data and pulse oximeter data.

2. A steering wheel felloe onto which the hands of the driver are placed during operation of the vehicle.

3. A sensor hub that is centrally positioned within the steering wheel and includes one or more sensor components.

4. Toggle buttons that provide directional control for the steering wheel sensory system if/when the directional controller is not included in the steering wheel.

5. A display screen, such as an LCD screen, LED screen, or OLED screen. The display screen is configured to visually output pulse, oxygen, and heart rate numbers detected by the steering wheel sensory system for benefit of the driver and/or passengers in the vehicle.

6. A camera that tracks the driver's facial status when driving and is communicably connected to the steering wheel sensory system.

7. An axle or spindle for rotating the steering wheel.

8. Wiring that interconnects the components of the steering wheel sensory system, providing control, power, and communicative channels between the components for operation of the steering wheel sensory system.

9. An integrated pulse oximeter and heart rate monitor sensor component that measures the oxygen level and heart rate of the driver.

10. A controller (or “control circuit”) that provides data processing of sensor data, bypass detection, and operates as the command center of the steering wheel sensory system.

11. A plurality of light photo sensors which capture sensor data including the heart rate and oxygen levels of the driver. After capturing the heart rate and oxygen levels of the driver, the light photo sensors send the sensor data to the controller of the steering wheel sensory system which determines whether to slow down and stop the vehicle (due to a detected cardiovascular health event) or allow the driver to continue driving as normal.

The various elements of the steering wheel sensory system may be related in the following exemplary fashion. It is not intended to limit the scope or nature of the relationships between the various elements and the following examples are presented as illustrative examples only.

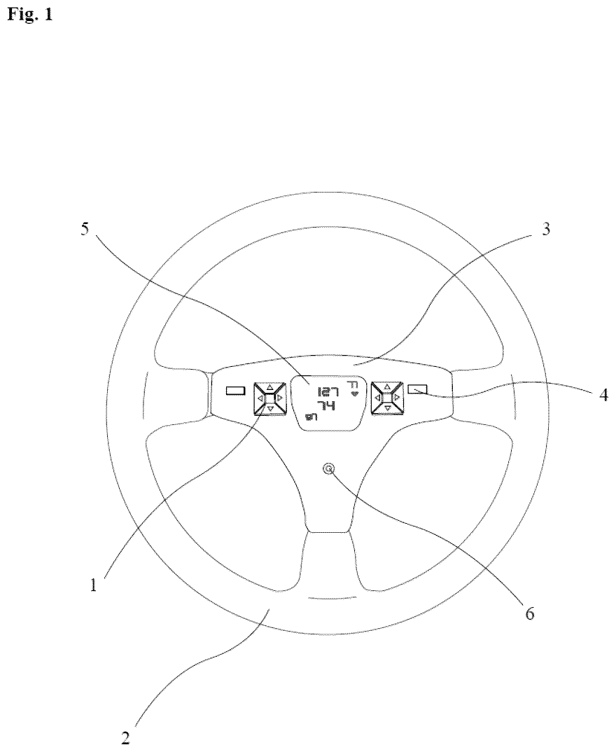

By way of example, FIG. 1 conceptually illustrates a front view of a steering wheel sensory system integrated into a first type of steering wheel. Specifically, this figure demonstrates a directional controller 10, a steering wheel felloe 20 of the first type of steering wheel, a sensor hub 30, a toggle button 40, a display screen 50, and a camera 60. There are two directional controllers 10 and two toggle buttons 40. The directional controllers 10 and the toggle button 40s are integrated on both right-of-center and left-of-center sides of the sensor hub 30, while the display screen 50 is positioned in the middle of the sensor hub 30 and the camera 60 is centered below the display screen 50. Surrounding the sensor hub 30 is the steering wheel felloe 20.

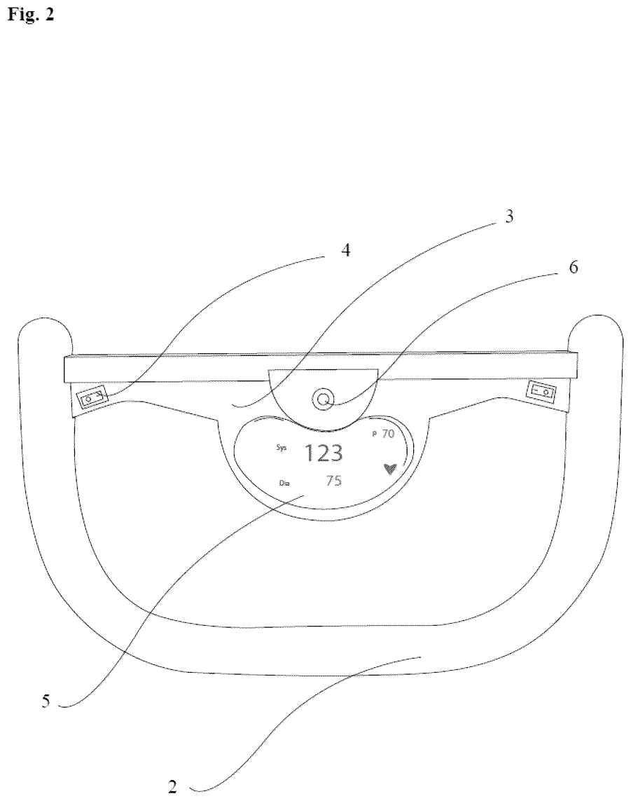

Turning to another example, FIG. 2 conceptually illustrates a front view of the steering wheel sensory system integrated into a second type of steering wheel. As shown in this figure, the second type of steering wheel does not include directional controllers 10, so toggle buttons 40 are available instead. Also, the sensor hub 30 has a different design form, due in part to the different shape and style of the steering wheel felloe 20. In addition, the display screen 50 fits prominently in the center of the sensor hub 30 with the camera 60 being disposed above the display screen 50 for this second type of steering wheel. As demonstrated in FIGS. 1 and 2, the display screen 50 is configured to visually output the sensor data captured by the steering wheel sensory system-specifically, pulse oxygen sensor data and heart rate sensor data. Also, the camera 60 is able to capture the driver's facial status when driving. The facial status may provide valuable information relating to various cardiovascular health events, such as stroke which could effect facial muscle coordination in ways that are indicative of a stroke or heart attack. Similarly, the camera 60 can capture other relevant health issues as they appear on the driver's face, such as lazy eye conditions in which one iris/pupil deviates from a field of vision that should be directed ahead toward the roadway, occlusion or obstruction of the eyes, impairing or preventing any vision by the driver of the roadway, etc. Accordingly, while the steering wheel sensory system focuses on driver safety by monitoring a driver's cardiovascular health state, the steering wheel sensory system is also capable of providing additional driver safety features.

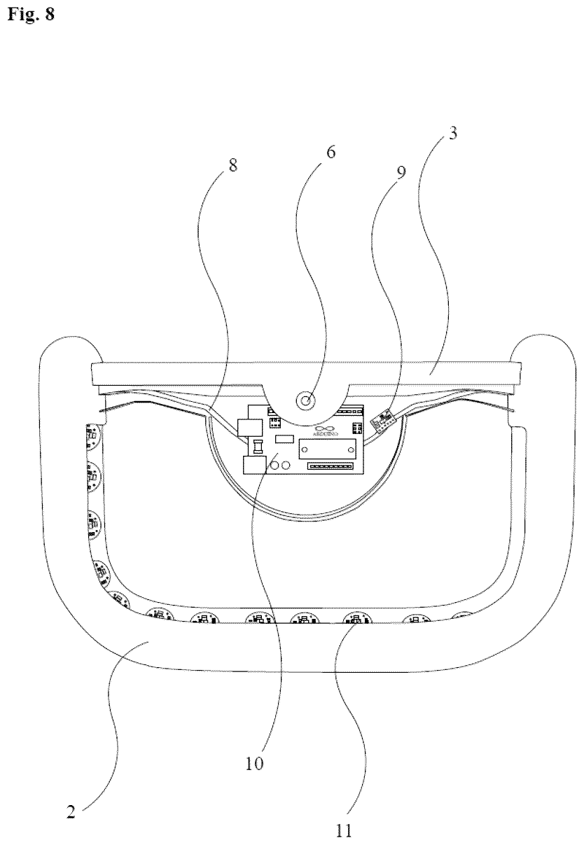

Notably, several sensors are embedded within the steering wheel felloe 20, but are not shown in FIG. 1 or 2. Also not shown in these figures are several embedded components within the sensor hub 30, including the controller. The embedded sensors, the controller, and the other embedded components are described below, by reference to FIG. 9.

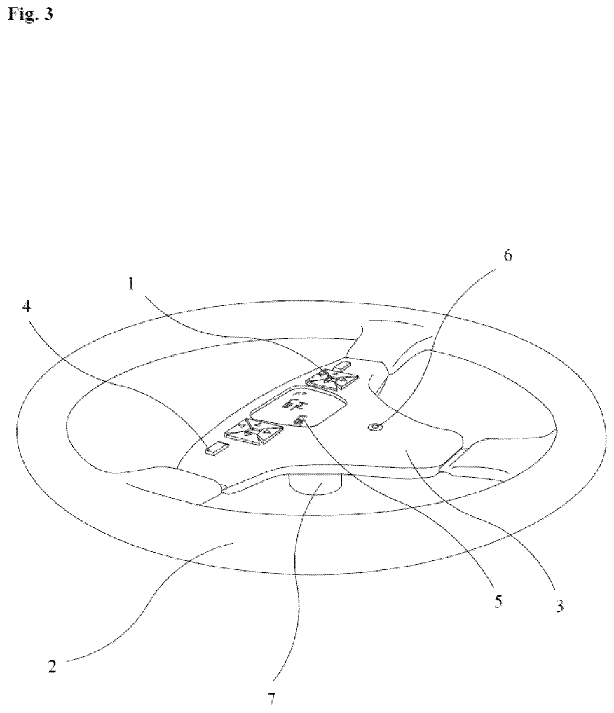

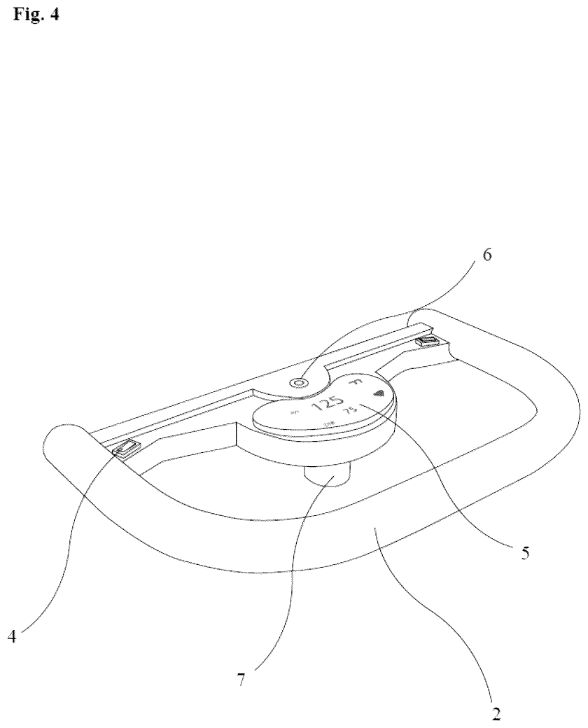

By way of example, FIGS. 3-4 conceptually illustrate side perspective views of the steering wheel sensory system integrated into the first type of steering wheel (for FIG. 3) and the second type of steering wheel (for FIG. 4). The additional component shown in these views is the axle/spindle 70 component for rotating the steering wheel. Typically, the axle/spindle 70 is housed withing a full steering column of the vehicle's steering wheel.

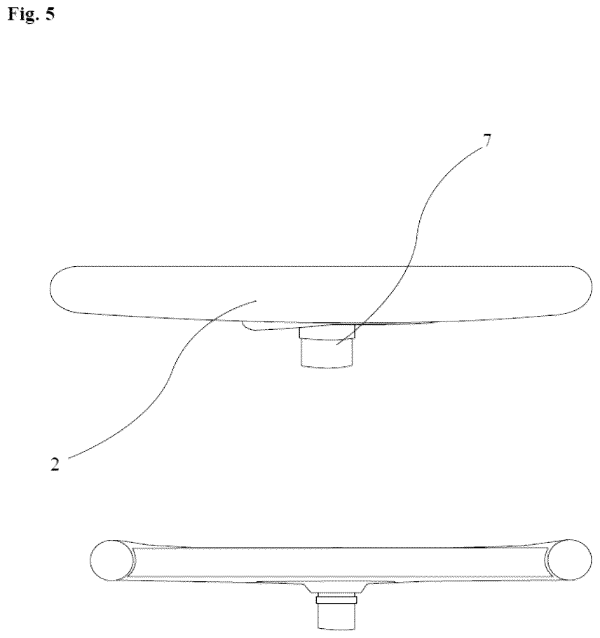

The steering wheel felloe 20 and axle/spindle 70 are further demonstrated in side views illustrated in FIG. 5, for the first type of steering wheel, and in FIG. 6, for the second type of steering wheel.

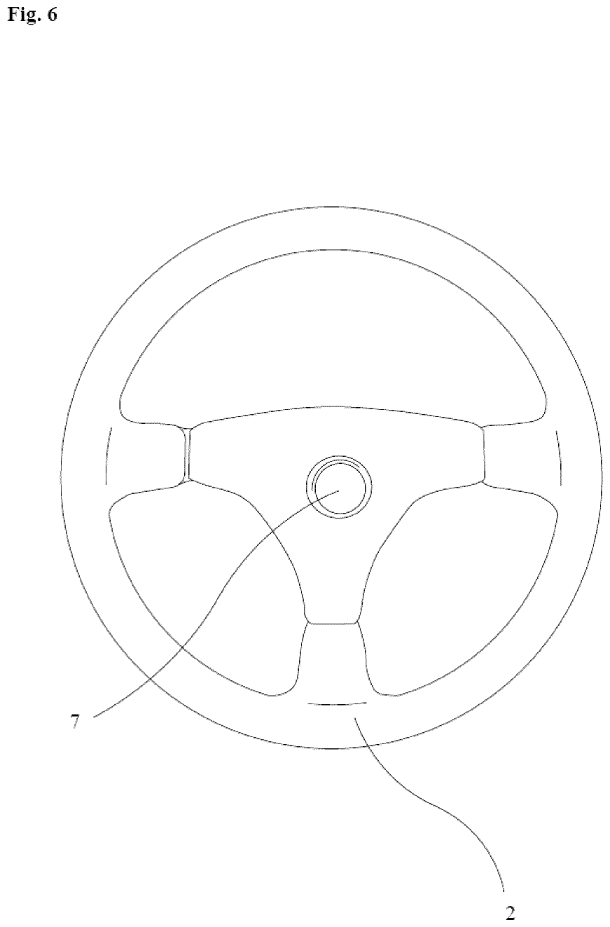

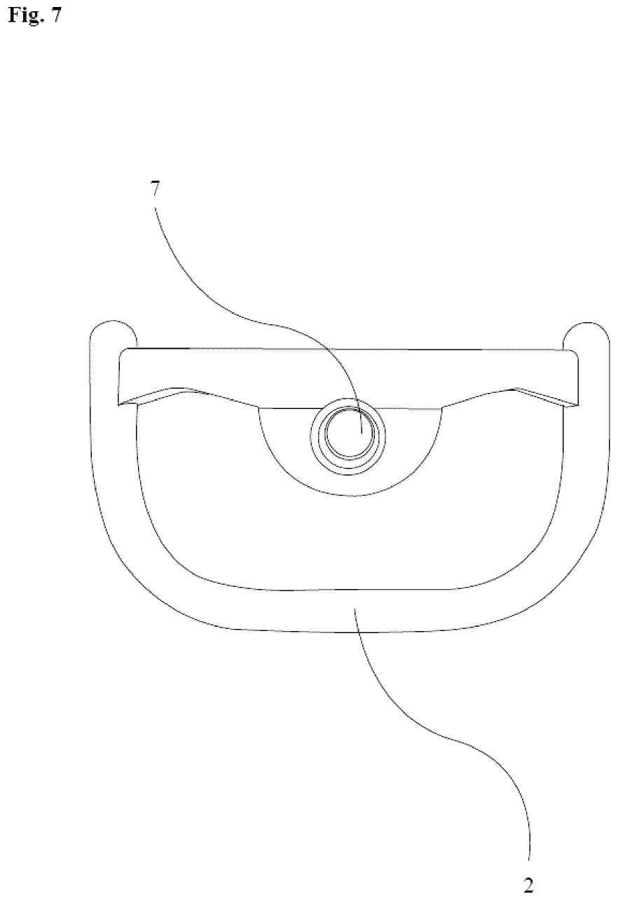

In addition to the views shown in FIGS. 5 and 6, the steering wheel felloe 20 and the axle/spindle 70 are further demonstrated in front views illustrated in FIG. 7, for the first type of steering wheel, and in FIG. 8, for the second type of steering wheel. Notably, certain elements are removed from these views to reduce those elements from obstructing the view of the axle/spindle 70 component. Specifically, the sensor hub 30, the directional controllers 10, the sensor hub 30, the toggle buttons 40, the display screen 50, and the camera 60 are removed from the view of the first type of steering wheel shown in FIG. 7. Similarly, the sensor hub 30, the toggle buttons 40, the display screen 50, and the camera 60 are removed from the view of the second type of steering wheel shown in FIG. 8.

By way of another example, FIG. 9 conceptually illustrates an interior view of the components of the steering wheel sensory system embedded in the second type of steering wheel. As shown in this figure, the second type of steering wheel incorporates the steering wheel felloe 20 having a U-shape (unlike the fully circular steering wheel felloe 20 incorporated into the first type of steering wheel). The sensor hub 30 and the camera 60 are also shown in this figure, but the display screen 50 and toggle buttons 40 are removed from view, which allows a view of the internal components which are those components embedded within the sensor hub 30 and within the steering wheel felloe 20. Specifically, the internal components shown in this view include the wiring 80, an integrated pulse oximeter and heart rate monitor sensor 90, and a PCB controller 100, all of which are embedded within the sensor hub 30. Additionally, the plurality of light photo sensors 110 are shown as embedded within the steering wheel felloe 20. Note that the PCB controller 100 may be any type of controller, micro-controller, computing device, single board computer, or other electronic system that is able to processor sensor data in real-time and trigger other systems to respond to cardiovascular health issues if and when they arise for a driver of a vehicle. Further details of the various types of electronic systems that may be deployed in and utilized by the steering wheel sensor system are described below, by reference to FIG. 10.

Turning back to the description of FIG. 9, the wiring 80 connects all of the components of the steering wheel sensory system, ultimately to the PCB controller 100. The key sensor components include the integrated pulse oximeter and heart rate monitor sensor 90 and the plurality of light photo sensors 110. The wiring 80 connects these sensors, as well as the camera 60, the display screen 50, and toggle buttons 40 (and/or directional controllers 10 when available) to the PCB controller 100 connecting to the bus for data throughput to the processing unit to perform the operations involved in monitoring the driver for health conditions that arise while driving, such as various cardiovascular health events, problems, issues, or risks that could render driving less safe than needed or, in the event of an actual, ongoing cardiovascular emergency, would render driving potentially fatal. Thus, the combination of components integrated into the steering wheel sensory system, as described above in connection with the first type of steering wheel, the second type of steering wheel, and any other type of steering wheel, are able to monitor and detect such risks and cardiovascular health events in real-time and automatically trigger the vehicle's ABS system to slow down and eventually stop the vehicle. Alternatively, monitoring the driver's cardiovascular health stats may proceed with no intervention at all, such as when the driver is in tip top cardiovascular shape, and thus, allow the driver to continue driving. In addition to the component-based description of the steering wheel sensory system described above, by reference to FIGS. 1-9, a more user-focused description of how the steering wheel sensory system is described next.

The steering wheel sensory system of the present disclosure generally works in the following manner. A driver enters the vehicle. The vehicle is equipped with a steering wheel that has embedded the steering wheel sensory system. To operate the vehicle, the driver would start vehicle ignition and place his or her hands on the steering wheel. Once hands are placed on the steering wheel, the sensor(s) of the steering wheel sensory system automatically initiate a sequence of steps for detecting heart rate of the driver. During this stage, the steering wheel is still locked (as if the vehicle were not operating). The steering wheel lock position is maintained by the steering wheel sensory system to give time for the initial heart rate to be detected. Once the heart rate of the driver is determined, if the heart rate of the driver is not a problem, the steering wheel sensory system releases the steering wheel lock. On the other hand, if the heart rate indicates a current cardiovascular health event, the steering wheel sensory system may not release the steering wheel lock.

Assuming at this point that the driver's baseline heart rate is in a range considered normal or in a range that does not indicate a present risk of a cardiovascular health issue, then the steering wheel sensory system unlocks the steering wheel. Once unlocked, the driver may start locomotive operation of the vehicle. During operation of the vehicle, the driver maintains his or her hands on the steering wheel. In this way, the steering wheel sensory system is able to continuously monitor the driver's heart rate, checking for erratic rhythms or other physiological markers of cardiovascular health problems.

In the event of a cardiovascular emergency, the steering wheel sensory system would detect the sudden change in heart rate and/or erratic rhythm. This triggers a cascade of safety measures starting with the sensors of the steering wheel sensory system communicating with the ABS sensors to initially slow the vehicle's speed down. Contemporaneously with slowing down the vehicle, the steering wheel sensory system notifies the driver of the elevated heart rate, erratic heart rhythm, or other physiological marker it detected (which indicates the driver may be experiencing a cardiovascular emergency). In addition to providing the information regarding the detected heart rate, rhythm, or other problem, the steering wheel sensory system also notifies the driver with instructions to pull over to the side of the roadway. In some embodiments, the steering wheel sensory system works in tandem with an AI system or a vehicle emergency system, such as OnStar® or other system, to contact emergency responders or medical assistance, if required or desired by the driver.

To make the steering wheel sensory system of the present disclosure, the sensors and various other components (such as communication systems, computing devices and/or printed circuit board (PCB) controllers, etc.) of can be implemented and embedded into the steering wheel when the steering wheel itself is being manufactured. Heart rate conditions can then be monitored on the vehicle dashboard or the steering wheel itself. The steering wheel sensory system would also be communicably connected to the vehicle's ABS systems and sensors.

Additionally, the steering wheel sensory system can be adapted to support navigation systems, emergency response systems (such as OnStar® or others), artificial intelligence (AI) and machine learning systems, etc.

To use the steering wheel sensory system of the present disclosure, a driver of a vehicle would need to place their hands on the steering wheel and start the ignition to initiate detection of a baseline heart rate of the driver. If and when the heart rate of the driver is satisfactory for driving, the steering wheel sensory system unlocks the steering wheel, thereby allowing the driver to operate the vehicle. The driver continues using the steering wheel sensory system as long as they maintain their hands on the steering wheel during operation of the vehicle. Note, the driver may use the steering wheel sensor system without actually driving anywhere. In other words, simply starting the ignition of the vehicle while their hands are placed on the steering wheel can provide valuable information to the driver about their heart rate-entirely without performing any locomotive operation of the vehicle, or even without unlocking the steering wheel, such as would happen if the steering wheel sensory system were to detect an abnormally high heart rate of the driver. Nevertheless, even with the steering wheel remaining in the locked position, the steering wheel sensory system would notify the driver via audible alarm and/or visual output on the vehicle dashboard or screen integrated into the steering wheel.

Many of the above-described features and applications are implemented as software processes that are specified as a set of instructions recorded on a computer readable storage medium (also referred to as computer readable medium, machine readable medium, or non-transitory computer readable medium). When these instructions are executed by one or more processing unit(s) (e.g., the process of the PCB controller 100, which may be a CPU, an AI processor, a GPU, a NPU, or may be a combination of multiple processors, cores of processors, or other processing units), they cause the processing unit(s) to perform the actions indicated in the instructions, including the monitoring of the sensor data and detection of sensor readings that suggest a cardiovascular health problem. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the terms “software”, “application”, and “program” (referred to below as “software”) are meant to include firmware residing in read-only memory or applications stored in magnetic storage, which can be read into memory for processing by a processor, such as the processor of a single board computer (SBC), a micro-controller, or other PCB controller board. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

By way of example, FIG. 10 conceptually illustrates the PCB controller 100 as an electronic system with which some embodiments of the invention are implemented. The PCB controller 100 may be a SBC, a controller or micro-controller, a mini-computer, or any other sort of electronic device with a control board capable of receiving and evaluating sensor data in real-time. Such an electronic system includes various types of computer readable media and interfaces for various other types of computer readable media. PCB controller 100 includes a bus 120, processing unit(s) 130, a system memory 140, a read-only memory 150, a permanent storage device 160, input devices 170, output devices 180, and a network 140.

The bus 120 collectively represents all system, peripheral, and chipset buses that communicatively connect the numerous internal devices of the PCB controller 100. For instance, the bus 120 communicatively connects the processing unit(s) 130 with the read-only memory 150, the system memory 140, and the permanent storage device 160.

From these various memory units, the processing unit(s) 130 retrieves instructions to execute and data to process in order to execute the processes of the invention. The processing unit(s) may be a single processor or a multi-core processor in different embodiments, and may be a specific type of processor, such as a CPU, a graphics processing unit (GPU), an AI processor, or a neural processing unit (NPU).

The read-only-memory (ROM) 150 stores static data and instructions that are needed by the processing unit(s) 130 and other modules of the electronic system. The permanent storage device 160, on the other hand, is a read-and-write memory device. This device is a non-volatile memory unit that stores instructions and data even when the PCB controller 100 is off. Some embodiments of the steering wheel sensory system use a mass-storage device (such as a magnetic or optical disk and its corresponding disk drive) as the permanent storage device 160.

Other embodiments of the steering wheel sensory system use a removable storage device (such as a floppy disk, an optical disk, or a flash drive) as the permanent storage device 160. Like the permanent storage device 160, the system memory 140 is a read-and-write memory device. However, unlike storage device 160, the system memory 140 is a volatile read-and-write memory, such as a random access memory (e.g., RAM, DRAM, DDRAM, etc.). The system memory 140 stores some of the instructions and data that the processor needs at runtime. In some embodiments, the invention's processes are stored in the system memory 140, the permanent storage device 160, and/or the read-only memory 150. For example, the various memory units include instructions for detecting cardiovascular health problems based on sensor data collected in real-time as a driver is driving the vehicle and, if the detected cardiovascular health is a serious and present danger, instructions for sending a command to the vehicle's ABS system to apply the brakes to slow down to a controlled and complete stop (i.e., the vehicle slowdown mechanism of the steering wheel sensory system). From these various memory units, the processing unit(s) 130 retrieves instructions to execute and the sensor data from the sensors to process in order to execute the real-time vehicular safety features of some embodiments.

The bus 120 also connects to the input and output devices 170 and 180. The input devices include the sensors. The input devices 170 may also include the directional controllers 10 and the toggle buttons 40 for making user selections of menu items visually output on the display screen 50. Similarly, the output devices 180 are those connected systems such as the display screen 50, as well as the vehicle's ABS system, emergency system (such as OnStar® or other), or other such output devices. In some embodiments, the display screen 50 is a touchscreen display which functions as both an input device and an output device.

Finally, as shown in FIG. 10, bus 120 also couples PCB controller 100 to a network 140 through a network adapter (not shown). In this manner, the PCB controller 100 can communicate with external systems (such as OnStar or other systems), or be a part of a network of computers (such as a local area network (“LAN”), a wide area network (“WAN”), or an intranet), or a network of networks (such as the Internet). Any or all components of PCB controller 100 may be used in conjunction with the invention.

These functions described above can be implemented in digital electronic circuitry, in computer software, firmware or hardware. The techniques can be implemented using one or more computer program products. Programmable processors and computers can be packaged or included in mobile devices. The processes may be performed by one or more programmable processors and by one or more set of programmable logic circuitry. General and special purpose computing and storage devices can be interconnected through communication networks.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

The above-described embodiments of the steering wheel sensory system are presented for purposes of illustration and not of limitation. While these embodiments of the steering wheel sensory system have been described with reference to numerous specific details, one of ordinary skill in the art will recognize that the steering wheel sensory system can be embodied in other specific forms without departing from the spirit of the invention. Thus, one of ordinary skill in the art would understand that the steering wheel sensory system is not to be limited by the foregoing illustrative details, but rather is to be defined by the appended claims.

I claim:

1. A steering wheel sensory system that is embedded in a steering wheel of a vehicle and detects heart rate of a driver when the hands of the driver are placed on the steering wheel, said steering wheel sensory system comprising:
a sensor hub of a steering wheel of a vehicle;
a printed circuit board (PCB) with a processor, a memory, and a sensor bus, wherein the PCB is embedded within the sensor hub of the steering wheel; and
an integrated pulse oximeter and heart rate monitor sensor that measures an oxygen level and a heart rate of a driver whose hands are placed on the steering wheel while operating the vehicle, wherein the integrated pulse oximeter and heart rate monitor sensor is embedded within the sensor hub of the steering wheel and is communicably connected to the PCB over the wiring which connects to the sensor bus.

2. The steering wheel sensory system of claim 1, wherein the PCB is communicably connected to both an anti-locking braking system (ABS) of the vehicle to provide a vehicle slowdown mechanism and a steering wheel locking system of the vehicle that maintains a wheel lock until baseline heart rate of the driver is detected via the integrated pulse oximeter and heart rate monitor sensor after ignition of vehicle.

3. The steering wheel sensory system of claim 1 further comprising a display screen integrated into and exposed along an exterior surface of the sensor hub and configured to visually output sensor data comprising pulse, oxygen, and heart rate values detected by the integrated pulse oximeter and heart rate monitor sensor.

4. The steering wheel sensory system of claim 3, wherein the display screen is further configured to visually output a menu of display options.

5. The steering wheel sensory system of claim 4, wherein the display screen comprises a liquid crystal display (LCD).

6. The steering wheel sensory system of claim 4, wherein the display screen comprises a light emitting diode (LED) display screen.

7. The steering wheel sensory system of claim 4, wherein the display screen comprises an organic light emitting diode (OLED) display screen.

8. The steering wheel sensory system of claim 4 further comprising a plurality of directional controllers integrated into the sensor hub of the steering wheel and configured to control the menu of display options visually output on the display screen.

9. The steering wheel sensory system of claim 4 further comprising a plurality of toggle buttons integrated into the sensor hub of the steering wheel and configured to provide directional control for controlling the menu of display options visually output on the display screen.

10. The steering wheel sensory system of claim 1 further comprising a camera integrated into and exposed along an exterior surface of the sensor hub and configured to track facial status of the driver when operating the vehicle.

11. The steering wheel sensory system of claim 1, wherein the sensor data bus comprises one of a Universal Asynchronous Receiver Transmitter (UART) bus, an Inter-Integrated Circuit (I2C) bus, a Serial Peripheral Interface (SPI) bus, and a Controller Area Network (CAN) bus.

12. The steering wheel sensory system of claim 1, wherein the PCB further comprises a wireless communication module.

13. The steering wheel sensory system of claim 12, wherein the wireless communication module comprises at least one of a Bluetooth® communications device, a WiFi device, a cellular communications device, and a global positioning system (GPS) device.

14. The steering wheel sensory system of claim 1, wherein the PCB comprises a single board computer (SBC) and the processor is integrated into the SBC.

15. The steering wheel sensory system of claim 14, wherein the processor comprises at least one of a central processing unit (CPU), a graphics processing unit (GPU), an artificial intelligence (AI) processor, and a neural processing unit (NPU).
